(19) United States
(12) Patent Application Publication
(10) Pub. No.: US 2024/0180431 A1
(43) Pub. Date: Jun. 6, 2024

(54) VENOUS TESTING AND VASCULAR ASSESSMENTS FROM INDUCED ANATOMICAL FLUID FLOW

(71) Applicant: Mayo Foundation for Medical Education and Research, Rochester, MN (US)

(72) Inventors: Steven E. Nowakowski, Rochester, MN (US); Thom W. Rooke, Rochester, MN (US); David A. Liedl, Eyota, MN (US); Valerie A. Vogt, Hayfield, MN (US)

(21) Appl. No.: 18/285,675

(22) PCT Filed: Apr. 19, 2022
(86) PCT No.: PCT/US2022/025301

§ 371 Date: Oct. 5, 2023

Related U.S. Application Data
(60) Provisional application No. 63/176,438, filed on Apr. 19, 2021.

(51) Int. Cl. A61B 5/02; A61B 5/00; A61B 5/026

(52) U.S. Cl. CPC A61B 5/02007; A61B 5/0053; A61B 5/02028; A61B 5/026; A61B 5/418; A61B 5/6828; A61B 5/7239; A61B 5/7282; A61B 2562/0247

(57) ABSTRACT

Systems, methods, devices, and other techniques for assessing a fluid system of a patient. A fluid evacuator applies compression to a cuff on a limb of the patient, and measures a pressure of the cuff during the compression to obtain a pressure response curve. The pressure response curve can be analyzed to identify an incompressible region, where the incompressible region is a portion of the pressure response curve indicative of a bodily fluid having been substantially evacuated from at least a portion of the limb as a result of the compression. Using information about the incompressible region of the pressure response curve, the system can at least one of (i) screen for one or more problems with the fluid system or (ii) evaluate an efficacy of the fluid evacuator in evacuating the bodily fluid from the limb of the patient

### CROSS-REFERENCE TO RELATED APPLICATIONS

This application claims the benefit of U.S. Provisional Application Ser. No. 63/176,438, filed Apr. 19, 2021. The disclosure of the prior application is considered part of (and is incorporated by reference in its entirety in) the disclosure of this application.

### BACKGROUND

### 1. Technical Field

This specification describes technology for assessing characteristics of fluid flow in a patient's anatomy (e.g., a circulatory or lymphatic system) by measurement of induced fluid flow.

### 2. Background Discussion

Much of the current testing of venous systems involves a patient performing toe tips while seated on the edge of an examination bed. In this testing, a measurement is made of the starting limb volume and the rate of volume increase following the completion of toe tips. Patient action is required to induce flow and no measurement is made while the flow is induced. However, patient action to induce flow can impair repeatability, consistent measurement and robustness in test results. Gravity can also be applied to induce flow. Gravity induced flow demands rapid patient manipulation which can be hazardous and tissue shifts may obscure measurements.

For example, some existing systems measure blood pressure (BP) using the DC method. The DC method determines the pressure where blood passes through an occluding cuff by detecting an increase in pressure of a sensing cuff. The increase in pressure is due to expansion of the limb under the sensing cuff due to blood inflow. The limb volume increase in diseased patients may be quite small. The small signal renders the DC method prone to noise and affected by volume and pressure levels in the sensing cuff. As the increase is gradual it can be difficult to define a point at which the increase starts.

Blood pressure has also been measured with an occluding cuff and a stethoscope. Systolic pressure is defined as the pressure where a stethoscope can detect the pulse distal of the occluding cuff. Pulse detection is specific rather than gradual making the measurement of systolic pressure more deterministic.

### SUMMARY

This specification describes systems, methods, devices, and other techniques for assessing characteristics of fluid flow in a patient's anatomy (e.g., a circulatory system such as a venous or arterial system, or a lymphatic system) by measurement of induced fluid flow. For example, embodiments of a venous testing system described in this specification can evaluate vascular function by applying one or more compression-decompression cycles to a limb to induce blood movement while simultaneously measuring the response. Implementations of the system can, in certain cases, provide relatively robust, quick, and inexpensive measurements, and the system may simply involve a blood pressure type cuff applied to a seated patient to execute a test protocol. The system can test for venous occlusion, venous insufficiency and poor pump function. More generally, the system can test for occlusion or insufficiency of any type of vessel such as veins, arteries, and/or lymphatic vessels. Evaluation can measure the functional effects of vessel disease (e.g., venous disease). The technologies described in this specification can be applied to venous testing, arterial testing, and/or lymphatic testing.

In general, one innovative aspect of the subject matter described in this specification can be embodied in methods for assessing a fluid system of a patient that include the actions of applying, with a fluid evacuation system, compression to a cuff on a limb of the patient, and measuring a pressure of the cuff during the compression to obtain a pressure response curve. The pressure response curve can be analyzed to identify an incompressible region, where the incompressible region is a portion of the pressure response curve indicative of a bodily fluid having been substantially evacuated from at least a portion of the limb as a result of the compression. Using information about the incompressible region of the pressure response curve, the system can at least one of (i) screen for one or more problems with the fluid system or (ii) evaluate an efficacy of the fluid evacuation system in evacuating the bodily fluid from the limb of the patient.

These and other implementations can further include one or more of the following features.

The limb can be a leg of the patient, the cuff can be disposed on a calf of the leg when the compression is applied, and the at least the portion of the limb can include the calf.

Analyzing the pressure response curve to identify the incompressible region can include identifying an inflection point in the pressure response curve that marks a transition from a compressible region of the pressure response curve to the incompressible region.

The compression can be applied to the cuff in a compression cycle, wherein the inflection point corresponds to a point in the compression cycle in which a predetermined amount of bodily fluid has been evacuated from the at least the portion of the limb.

The method can include screening for an obstruction in the fluid system based on a pressure value at the inflection point. A line can be fitted to the incompressible region of the pressure response curve. Using the line, an amount of bodily fluid evacuated from the at least the portion of the limb during the compression can be determined.

The method can include screening for an obstruction in the fluid system based on an absence in the pressure response curve of an inflection point between a compressible region of the pressure response curve and an incompressible region of the pressure response curve.

The compression can be applied to the cuff in a first compression-decompression cycle, and the method can include: applying compression to the cuff in a second compression-decompression cycle following the first compression-decompression cycle; determining a slope of a second incompressible region in the pressure response curve, wherein the second incompressible region occurs during compression in the second compression-decompression cycle; determining a slope of a pre-evacuation region in the pressure response curve, wherein the pre-evacuation region precedes the second incompressible region during compression in the second compression-decompression cycle; comparing the slope of the pre-evacuation region to the slope of the second incompressible region; and assessing vessel insufficiency based on a result of comparing the slope of the pre-evacuation region to the slope of the second incompressible region.

The method can include applying decompression to the cuff following the compression, including: decompressing the cuff in a first phase from a peak pressure to a first reduced pressure that is less than a defined amount of column height pressure; pausing, for a first paused interval, the decompression after the first phase upon reaching the first reduced pressure; decompressing the cuff in a second phase from the first reduced pressure to a second reduced pressure that is below the column height pressure; pausing, for second paused interval, the decompression after the second phase upon reaching the second reduced pressure; decompressing the cuff in a third phase from the second reduced pressure to a third reduced pressure; measuring the pressure of the cuff during the decompression, including during the first phase, the second phase, and the third phase, and augmenting the pressure response curve with a pressure response measured during the decompression; and assessing vessel insufficiency based on characteristics of the pressure response curve during at least one of the first paused interval or the second paused interval.

The compression can be applied to the cuff in a first compression-decompression cycle, and the method can include: applying compression to the cuff in a second compression-decompression cycle following the first compression-decompression cycle, wherein the compression in the second compression-decompression cycle is initiated before the bodily fluid substantially refills in the limb; measuring the pressure of the cuff during the compression in the second compression-decompression cycle, and augmenting the pressure response curve with a pressure response measured during the compression in the second compression-decompression cycle; and assessing ejection function of a heart of the patient based on at least one characteristic of the pressure response measured during the compression in the second compression-decompression cycle.

Applying compression to the cuff can include pumping a working fluid into the cuff at a constant rate, wherein the working fluid is compressed air, oxygen, or water.

The fluid system can be a venous system and the fluid is blood, the fluid system can be an arterial system and the fluid is blood, or the fluid system can be a lymphatic system and the fluid is lymph.

Other embodiments of this aspect include corresponding computer systems, apparatus, and computer programs recorded on one or more computer storage devices, each configured to perform the actions of the methods. For example, the aspect can include one or more computer-readable storage media encoded with instructions that, when executed by one or more computers, cause the one or more computers to perform any of the methods and processes described in this specification. In some implementations, a system includes one or more computers and one or more computer-readable storage media encoded with instructions that, when executed by the one or more computers, cause the one or more computers to perform any of the methods and processes described in this specification.

In general, one innovative aspect of the subject matter described in this specification can be embodied in a system, comprising: a pump configured to increase pressure of a working fluid in a cuff during a compression action; a valve configured to release pressure from the cuff during a decompression action; a pressure transducer configured to measure a pressure of the working fluid in the cuff; and one or more controllers configured to actuate the pump and the valve to control a level of pressure of the working fluid in the cuff via compression and decompression actions based at least in part on measurements from the pressure transducer.

These and other implementations can further include one or more of the following features.

The system can further include the cuff.

The system can further include a flow meter configured to measure a flow of the working fluid into or out of the cuff, wherein the one or more controllers are further configured to control the level of pressure of the working fluid in the cuff based on measurements of working fluid flow from the flow meter.

The system can further include one or more bodily fluid sensors configured for placement on a limb of a patient in a region of the limb separate from the cuff, wherein the system is further configured to measure a level of flow of a bodily fluid in the patient using signals from the one or more bodily fluid sensors, wherein the bodily fluid is blood or lymph.

Other embodiments of this aspect include corresponding computer systems, apparatus, and computer programs recorded on one or more computer storage devices, each configured to perform the actions of the methods.

The details of one or more embodiments of the subject matter of this specification are set forth in the accompanying drawings and the description below. Other features, aspects, and advantages of the subject matter will become apparent from the description, the drawings, and the claims.

### BRIEF DESCRIPTION OF THE DRAWINGS

FIG. 1 is a diagram of an example venous testing system connected to a cuff worn on a calf of a patient.

FIG. 2 is a plot of an example pressure response curve.

FIGS. 3A and 3B depict plots of cuff pressure versus time and volumes of cuff and limb versus time, respectively.

FIG. 4 is a diagram of an example venous testing system.

FIG. 5 depicts diagrams of a hydraulic model of a venous system of the leg.

FIG. 6 depicts an example pressure response curve over a compression-decompression cycle.

FIG. 7 is a plot depicting the pressure versus time relationship to bladder volume and blood flow.

FIG. 8 is a diagram illustrating inflation volume and pressure by injecting a fluid (e.g., air) into a container at a constant rate.

FIG. 9 is a plot of an example pressure response curve augmented with xlength calculation.

FIG. 10 is a plot of an example pressure response curve for a patient without venous occlusion.

FIG. 11 is a plot of an example pressure response curve for a patient with minor venous occlusion.

FIG. 12 is a plot of an example pressure response curve for a patient with significant venous occlusion.

FIG. 13 is a plot of an example pressure response curve for a patient with venous insufficiency.

FIG. 14 is a plot of a decompression method for detecting venous insufficiency.

FIGS. 15A-15C depict pressure response curves in relation to detection of venous insufficiency through a decompression method.

FIG. 16 depicts an example pressure response curve for an empty leg sequence.

FIGS. 17A-17B depict example pressure response curves reflecting good and poor pump function, respectively.

FIG. 18 depicts a pressure response curve for an example testing protocol sequence.

FIG. 19 is a diagram of an example venous testing system.

FIG. 20 is a flowchart of an example process for assessing obstruction of a venous system of a patient.

FIG. 21 is a flowchart of an example process for estimating an amount of blood evacuated from a limb of a patient over a compression cycle.

FIG. 22 is a flowchart of an example process for assessing obstruction of a venous system based on absence of an inflection point during compression.

FIG. 23 is a flowchart of an example process for assessing venous insufficiency of a patient.

FIG. 24 is a flowchart of an example process for assessing venous insufficiency of a patient.

### DETAILED DESCRIPTION

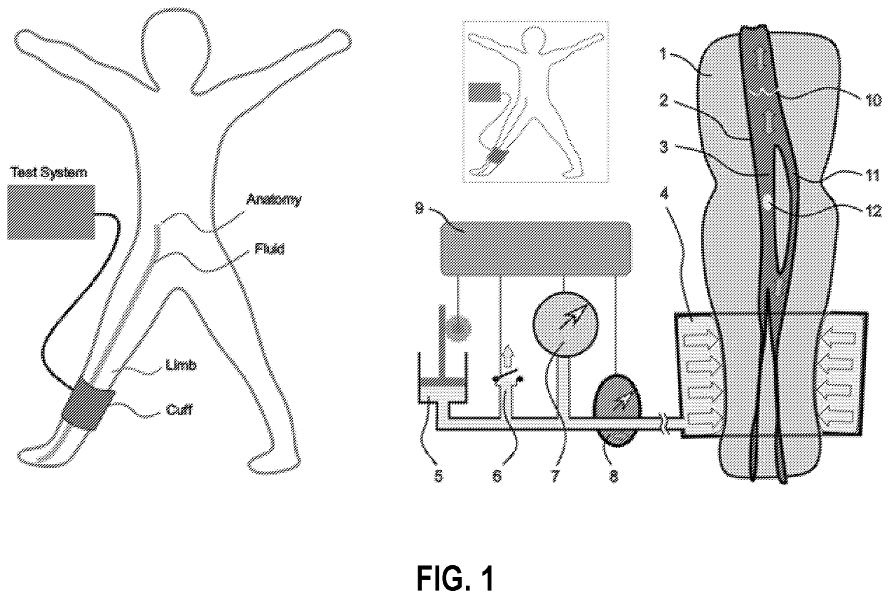

FIG. 1 depicts a venous testing system according to an embodiment of the techniques described herein. The system is configured to actively induce fluid movement in a patient limb while simultaneously measuring pressure and flow in order to assess characteristics of the anatomical system supporting flow of the fluid. A cuff (4) wrapped around the patient limb (1) applies cycles of compression and decompression. The rates of both compression and decompression are controlled, along with the times between compression-decompression cycles. Measurements of fluid movement are captured either from the cuff (4) applying the compression or from other sensors attached to the limb (1). Measurements of flow are correlated with the applied compression/decompression to evaluate flow characteristics of the fluid system.

For example, low flow under high compression force can indicate an obstruction in the fluid system. Flow characteristics of the anatomy can include overall rate of flow, blockages, failed/operational valves, amount of fluid in a limb, etc. Any limb can be tested by the system (e.g., left leg, right leg, left arm, right arm, individually or together). Several different fluid systems can be evaluated such as venous, arterial or lymphatic systems. Characteristics of the flow system beyond the limb being compressed can be evaluated. For example, venous flow through the thigh and into the lower abdomen can be evaluated via compression and measurement at the calf. Flow in multiple directions can be evaluated such as venous reflux with a series of compression and decompression cycles.

The systems, methods, devices, and techniques disclosed herein “test” flow characteristics of the fluid in an anatomical system (e.g., blood in a venous system). The anatomical systems to be evaluated often contain multiple channels and active features such as valves. When the test system is applied for evaluation of the venous system, venous blood is induced to flow at a controlled rate in order to measure the ability for the veins to carry flow. A high resistance to flow indicates obstruction in the veins which has not been reduced by redundant pathways.

Decompression of the limb is an active mode of flow. Compression can empty fluid from the limb in a substantially thorough and consistent manner. Release of the compression (decompression) allows inflow of fluid. Inflow can be evaluated for rates consistent with healthy or unhealthy processes. In the case of blood flow, inflow into a limb can occur by arterial flow or by reverse venous flow through failed valves. A patient can be positioned so that gravity will induce reverse flow (e.g., seated). The rate of inflow during decompression can detect reverse flow indicating valve failure.

In FIG. 1, an apparatus is shown that drives fluid movement in a patient limb and measures the rate of flow versus pressure and time in order to assess functional characteristics of the anatomical system supporting flow of the fluid. The apparatus includes a pneumatic cuff (4) wrapped around a patient limb (1). The cuff is inflated by a pump (5) and deflated by a valve (6). Pressure and flow in the system are measured by a pressure transducer (7) and flow meter (8). A control system (9) operates the pump and valve to produce regulated inflation and deflation rates. The control system also captures cuff pressure and flow measurements versus time. The cuff applies compression to the limb, squeezing the anatomical structures and thereby producing fluid flow out of the limb while release of the compression allows flow into the limb. Sequences of compression and decompression support testing of flow out of and into the limb. Functional flow characteristics evaluated by the apparatus include failed or operational valves (10), resistance to flow from an obstruction (11), volume of fluid in the limb, or rate of fluid inflow to the limb from external processes. Conditions such as obstructive disease, venous reflux, or poor pump function can be diagnosed from functional test results. Fluid systems which could be evaluated include venous, arterial or lymphatic systems. Any limb could be tested by the apparatus. Characteristics of the anatomical system beyond the limb wrapped by the cuff can be evaluated by the apparatus if the fluid channel continues further beyond the limb. Testing limbs simultaneously can amplify the effect on proximal branches of the anatomical fluid system due to the tree like structure. For example, venous flow through the thigh and into the lower abdomen could be evaluated via simultaneous compression and measurement at the left and right patient calves.

The anatomical system that supports fluid flow can include obstructions. An obstruction may or may not functionally block fluid flow depending on degree of blockage and whether alternate paths exist. The apparatus can apply compression to force fluid to be ejected from the limb and the pressure at which the fluid is ejected indicates resistance to flow. High resistance indicates a functional obstruction in the channel.

The anatomical system can include valves which, if operational, allow fluid flow in only one direction. Reverse flow occurs when a valve fails. Flow into the limb from normal inflow or abnormal reverse flow can be measured by monitoring the expansion of an empty limb. Decompression and/or passive measurement after a compression by the apparatus can allow for measuring expansion from inflow. The timing of inflow can also indicate whether the source is normal inflow or reverse flow.

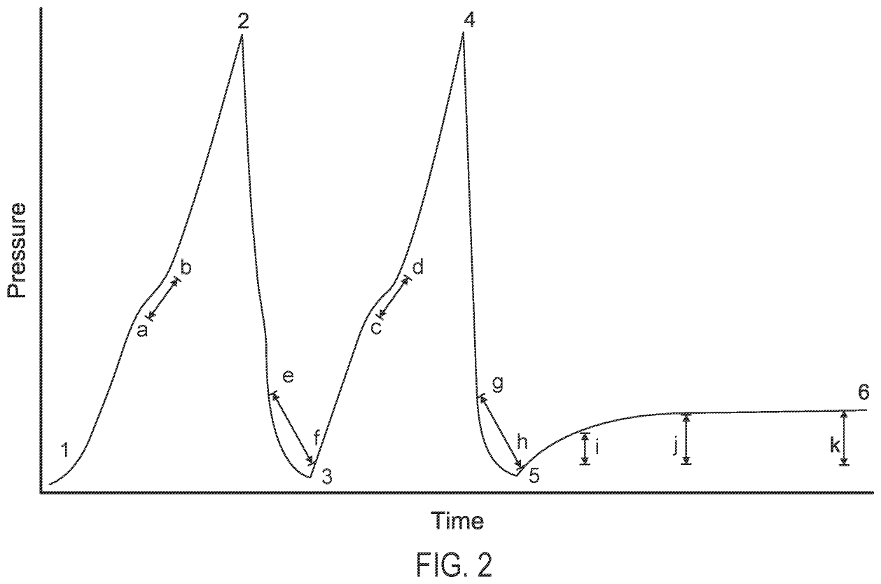

In some implementations, the systems and techniques described herein provide three operational modes: compression, decompression, and passive. The controller captures measurements in all modes (e.g., pressure and/or flow measurements). In compression mode, the pump adds a constant volume of air (or other medium such as water) at room pressure to the cuff. In decompression mode, air in the cuff is released at a controlled rate by the exhaust valve. In passive mode the pump is off and the valve is closed so that no air flows in or out of the cuff. FIG. 2 illustrates pressure data for a typical sequence of modes.

Compressions (corresponding to regions 1 to 2 and 3 to 4 in FIG. 2) can serve a number of functions including: enabling measurement of characteristics related to forward flow by inducing fluid flow out of the limb; creating the conditions for measuring characteristics of the anatomy related to in-flow by substantially emptying fluid from the limb; compressions can be repeated to ensure a fully empty limb; and to measure the amount of fluid held in the limb. Decompressions (corresponding to regions 2 to 3 and 4 to 5 in FIG. 2) serve a number of purposes including creating conditions for fluid flow into the limb and observation of initial fluid inflow. When the compression force falls below the force inducing inflow, fluid flow will commence. Decompression can also be a required transition to passive mode or to another compression. Passive mode (corresponding to regions 5 to 6 in FIG. 2) can be used to observe flow into the limb over longer periods of time or inflow which occurs at pressures consistent without applied compression.

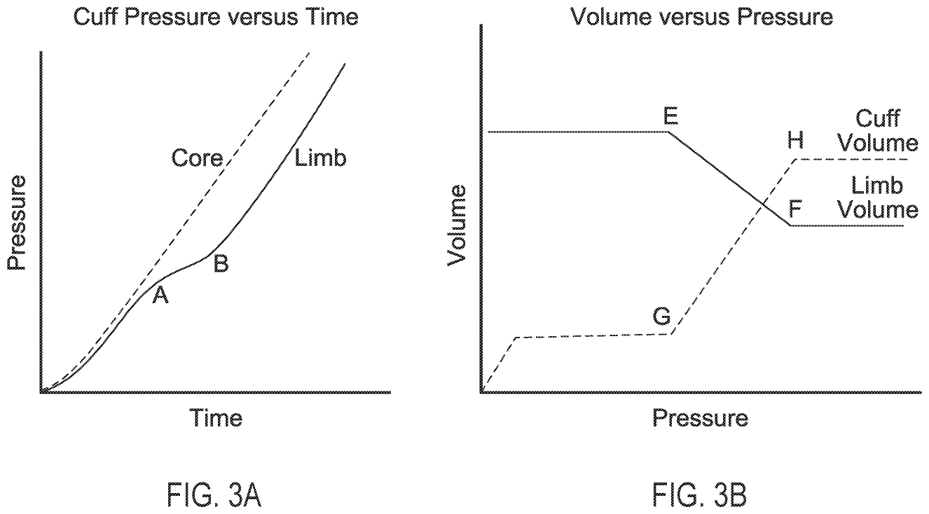

FIG. 3B illustrates that as pressure in the cuff increase the volume of the cuff and the limb are constant until fluid is ejected from the limb. When fluid moves out of the limb, the limb decreases in volume which allows the cuff wrapped around the limb to increase in volume (E to F in FIG. 3B). Volume in the cuff is inverse to limb volume (G to H in FIG. 3B). Stated differently, change in cuff volume is inverse to the rate of fluid moving out of the limb.

FIG. 3A illustrates typical data for a compression cycle. The Core line shows data for a cuff applied to an incompressible core. If the cuff does not change in volume the pressure in the cuff increases nearly linearly with time. The Limb line in FIG. 3A shows data for a cuff on a limb. The limb data shows a region of reduced slope (A to B in FIG. 3A). The region of reduced slope is where fluid moves out of the limb. An increase in the cuff volume causes a decrease in cuff pressure per Boyles Law (P1*V1=P2*V2). The cuff volume increase does not appear in the data curve as a reduction in pressure but rather as a decrease in the rise of pressure because the apparatus is continuously adding air to the cuff. The controller detects the pressure range of decreased slope. This range is when the fluid flows. The pressure at which the fluid is ejected gauges the resistance to flow in the fluid anatomy (Also shown in FIG. 3 curve sections a to b and c to d). For reference, a pressure response curve for a compression cycle like that shown in FIG. 3A can generally be referred to as having three regions: (1) a “pre-flow” region (from the start of the compression cycle to point A in FIG. 3A), (2) an “active flow” or “compressible” region (from points A to B in FIG. 3A), and (3) a “post-flow” or “incompressible” region (from point B to the end of the compression cycle in FIG. 3A). The inflection points A and B between these regions can be defined in any suitable manner that delineates the reduced slope of the active flow region from the steeper slopes of the pre-flow and post-flow regions. For example, the rate of change or acceleration of the slope changes can be evaluated to determine inflection points, or the point closest to an intersection of lines fitted to each region can be selected as the inflection point. Inflection points can also be related to points where a defined amount of blood (or other fluid) has evacuated the limb due to compression. For instance, the first inflection point A can be based on an estimation that ten percent of the fluid has evacuated the limb, and the second inflection point B can be based on an estimation that eighty or ninety percent of the fluid has evacuated the limb.

Decompression and passive modes observe fluid flowing into a limb after the limb has been emptied by a compression. During a decompression pressure on the limb is quickly reduced. Inflow will occur when the pressure applied by the cuff drops below the pressure of the force causing inflow. Inflow during decompression causes the pressure to decrease more slowly. This would be observed in regions e to f and g to h of FIG. 2.

Passive mode allows observation of inflow at a low rate, under typical conditions and for longer periods of time. In passive mode, the pressure increase is analyzed based on time to reach 50% and 90% of endpoint pressure (points i, j and k respectively in FIG. 2). Because cuff and limb volumes are patient dependent a relative measurement of the inflow is appropriate. Criteria can be established such as: “the time to 90% of the endpoint should be greater than 30 seconds”.

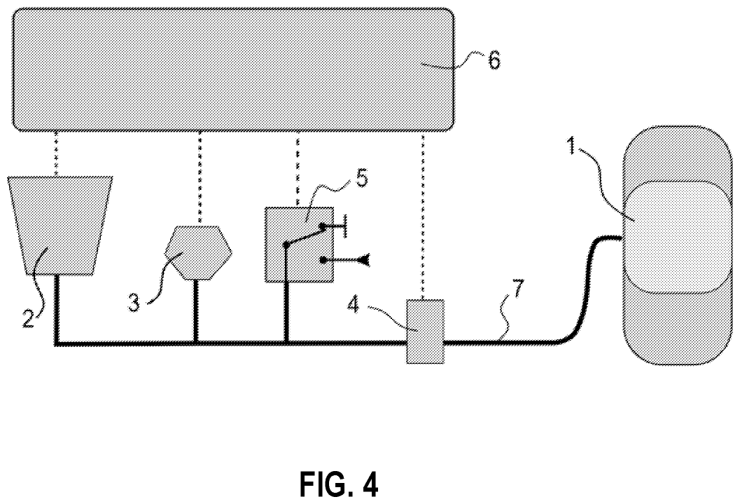

FIG. 4 depicts a block diagram of an example system or apparatus comprising a pneumatic cuff (1) wrapped around a patient limb to apply compression in order to produce fluid flow in the supporting anatomical system. The cuff is inflated by pump (2) and deflated by exhaust valve (5). Both pump and valve are operated by controller (6). Controller (6) captures and stores pressure measurements from pressure transducer (3). The controller (6) also captures and stores flow measurements from flow sensor (4). A pneumatic or fluid circuit (7) connects pump output (2), valve (5), and pressure transducer (3) to the cuff (1). The circuit supports air flow into and out of the cuff and provides equal pressure to all connected items. Air to or from the cuff flows through flow sensor (4). The controller (6) regulates the rate of inflation and deflation by controlling the pump (2) and exhaust valve (5). The controller may execute a timed sequence of compressions, decompressions and passive (no inflation or deflation) modes. The controller can be implemented using one or more hardware circuits and/or processors in one or more locations. In some implementations, functionality of the controller can be split among multiple devices, such as a first device that controls the pump and exhaust valve, a second device that measures signals from flow and pressure sensors, and a third device that analyzes the measurements (e.g., pressure response curves) to generate outputs indicating the presence, absence, or likelihood of conditions such as obstruction or anatomical valve of pump problems.

With reference to FIG. 4, in some implementations, the apparatus implements compression mode to force fluid to flow out of the limb via the anatomical system and is created by inflating the cuff at a controlled rate from pump. The apparatus implements decompression to allow fluid to flow into the limb via external forces and is accomplished by stopping the pump (2) and opening valve (5) to release air. The apparatus implements passive mode to allow fluid to flow into or out of the limb via external forces and is accomplished by stopping the pump (2) and closing valve (5) to hold air in the cuff. In all test modes pressure transducer (3) measures pressure in the system which is equal to pressure in the cuff. In all test modes flow meter (4) measures air flow in the system which is equal to flow into or out of the cuff. Controller (6) implements can implement routines to regulate the pump (2) to produce a controlled rate of inflation. Controller (6) can implement routines to regulate the valve (5) during decompression to produce a controlled rate of deflation. The controller can also close the valve (5) to allow for inflation of the cuff. The controller can analyze the captured pressure and flow data to detect when the fluid moves out of the limb under compression by isolating the pressure range of reduced pressure increase per time increment (e.g., identify the “active flow”/“compressible” region in the pressure response curve). Alternatively, the flow meter data can indicate a pressure range of increased flow per pressure increase. A patient can be positioned so that forward fluid flow works against gravity. Compression must apply a pressure to overcome the gravitational force before the fluid will move. Thus, the expected pressure range for fluid movement can be manipulated into a range where the apparatus is most effective.

Normal inflow to a limb can also be measured on an empty limb by utilizing a compression to empty the limb followed by a decompression and passive mode can detect inflow into the limb. The data can be analyzed to compare endpoint volume versus time to 50% of endpoint and 90% of endpoint volume. A patient can be positioned so that forward fluid flow works against gravity. Compression can empty the limb then a decompression followed by a passive mode can detect inflow into the limb. Reverse flow induced by gravity can measure reverse flow enabled by features such as a failed valve. The controller can analyze the pressure versus time data for decompression and detect a pressure range where pressure decrease versus time decreases less than a defined (e.g., threshold) amount indicating an inflow of fluid at that pressure.

The apparatus can be implemented as part of a venous thrombosis prevention system to measure the efficacy of the venous blood pumping. The apparatus can execute a diagnostic inflation immediately following a therapeutic inflation to measure the amount of blood in the limb after therapeutic inflation. The amount of blood is relative to the pressure range over which reduced pressure increase is observed. A flow meter can be employed to measure the volume of blood movement. The apparatus can be implemented as part of a venous thrombosis prevention system to provide early detection of thrombosis. The apparatus can detect elevated pressure required to force fluid out of the limb indicating a developing thrombosis.

### Model of the Veins as a Hydraulic System

The veins of a leg form a system of serial and parallel channels whose fundamental function is to return blood to the heart. The serial and parallel channels provide redundancy and aggregation of flow. Valves in the veins prevent backward flow of the blood. Muscle movement around the veins forces blood out of the local vein region and by operation of the valves pumps blood toward the heart. Obstructions in the veins impede flow of blood toward the heart causing blood to pool in the leg causing swelling and other issues. Valves both support forward blood flow toward the heart while preventing backwards flow. Blood flow toward the heart should operate in a ratchet like manner where each muscle contraction of the calf lifts blood past a system of valves marching to the heart.

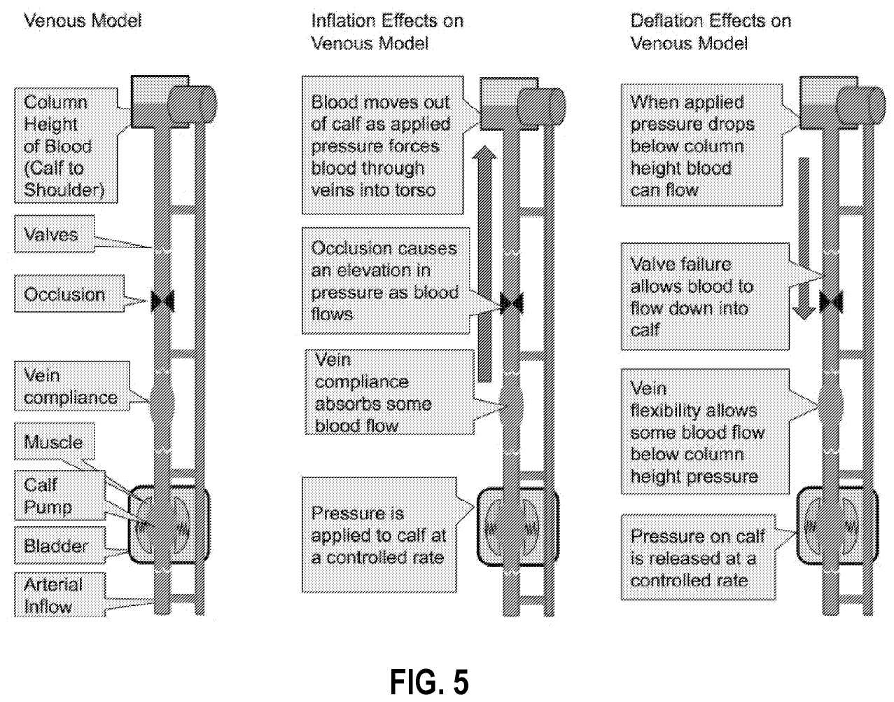

Valve breakdown prevents pump function from accomplishing blood movement while also allowing backward flow of blood. Pump function can be ineffective outside of valve failure though the mechanism is not well understood. The venous system contains multiple paths and multiple active components but is inherently a hydraulic system. This hydraulic system is charged with returning blood to the heart. Consequently, evaluation of the health of the venous system can be based on evaluation of blood flow. FIG. 5 depicts a hydraulic model of the venous system of a leg of a patient, for example. As used in this specification, the term “patient” should be understood broadly to encompass any person or individual on which measurements are performed, regardless of whether the person or individual is receiving clinical treatment or evaluation.

Blood is the ‘working fluid’ of the venous testing system. The venous system is the routes formed by the veins, the valves controlling direction of flow, and the pump function from muscles contracting on the veins. A hydraulic system cannot be tested empty. A ‘working fluid’ is forced through the system while characteristics like direction, flow rate at pressure are measured.

The air bladder in a cuff wrapped around the limb both applies compression force and measures the effect of the compression. As the bladder is inflated it applies compression to the limb. The bladder is in intimate contact with a significant section of the limb. A pneumatic cuff can apply stimulus to the body while capturing measurements. Air is pumped into the bladder to apply compressive force to the limb or air is released to decompress the limb. The air volume added to the bladder by the venous testing system is controlled while the response of the limb in the form of pressure achieved in the bladder is measured.

Suitable regions of the leg can be compressed to force blood flow out of the limb. Release of the compression (decompression) allows the reverse flow of blood. Gravity also can be used to induce blood flow by positioning the patient so the limb is below the torso. Gravity induces not only reverse blood flow but also resistance to forward flow. The venous system in the leg must overcome gravity to move blood toward the heart whenever the torso is above the leg. Blood in the veins forms a column of fluid from the shoulder down to the leg. This force of this column has a significant effect on the movement of venous blood.

Blood can be forced out of a limb through compression at any location (e.g., thigh, calf). A larger amount of blood movement provides more range for the dynamic testing. A location on the limb with more venous blood available can provide more time for measuring system response. The calf contains a large amount of blood in a rested patient, and is preferably used in some applications. Moreover, the calf is relatively cylindrical and not overly large allowing more effective coupling to a cuff and bladder for compression and pressure measurement.

### Measuring Blood Movement Indirectly

Blood flows through the veins eventually returning to the heart, but veins are elastic expanding or contracting to thereby containing more or less blood. The limb expands in volume with the net flow of blood into the veins while a net flow of blood out of the veins results in a contraction of limb volume. Limb contraction and expansion can be tracked to indirectly measure the flow of blood into or out of a limb. An air filled bladder is wrapped around a limb contained by an inelastic cuff. When the limb shrinks in volume the bladder expands and vice versa. The expansion or contraction of the bladder is equal and opposite of the movement of the limb. Thus the volume change in the bladder is equal and opposite of the volume change of blood in the limb.

The air bladder follows pneumatic rules. If the amount of air in the bladder is held constant (no air flow in or out), as the volume of the bladder changes the pressure changes inversely. By measuring the pressure change in the bladder, limb expansion or contraction can be tracked thereby indirectly measuring blood movement.

### The Incompressible Line

A limb becomes relatively incompressible after the majority of blood is forced out of the limb. This “incompressible” or “post-flow” region allows the venous testing system to establish a useful baseline for measurements. Since the limb is not expanding or contracting the bladder holds a substantially stable volume even though pressure in the bladder is changing. The pump is injecting a constant volume of air at room pressure into the bladder throughout the compression. The incompressible region creates a linear relationship for pressure versus time for the unchanging system. The incompressible line becomes a baseline for evaluating pressure measurements made while blood is flowing in or out of the limb.

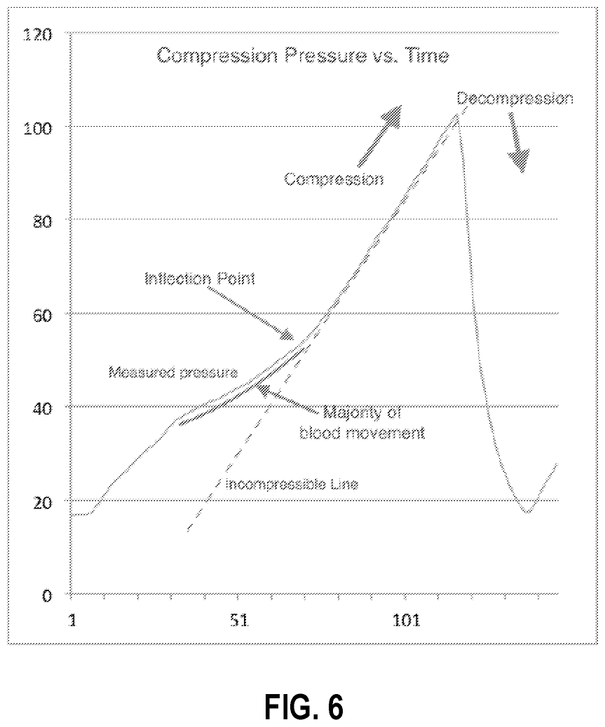

FIG. 6 shows measured data for a healthy patient calf during a compression cycle. The blood is forced out of the limb through application of compression. The majority of blood moves when the compression force overcomes the force of gravity and completes at the inflection point equal to the force from the height of fluid from calf to shoulder. At compression pressures above the inflection point (e.g., 60 mmHg) venous blood has been forced out of the limb and a substantially linear relationship between pressure and time begins in the post-flow or incompressible region. The venous testing system fits a line to this region typically using pressures from 80 to 100 mmHg for fitting. This pressure versus time line can then be extended to lower pressures (earlier time) to use as a baseline for several measurements.

The incompressible line can be used to detect when the blood has been forced out of the limb. During compression when the measured pressures conform to the incompressible line the blood has been forced out of the limb. A linear line fit to the data points from 80 to 100 mmHg can define the incompressible line, for example.

Blood has been forced out of the limb when the measured data points begin to conform to the incompressible line. The time and pressure at this point is considered to be an inflection point. The pressure at this point can indicate the presence or absence of obstruction while the time at this point can be used to discern the volume of blood forced out of the limb.

### Measurement During Compression

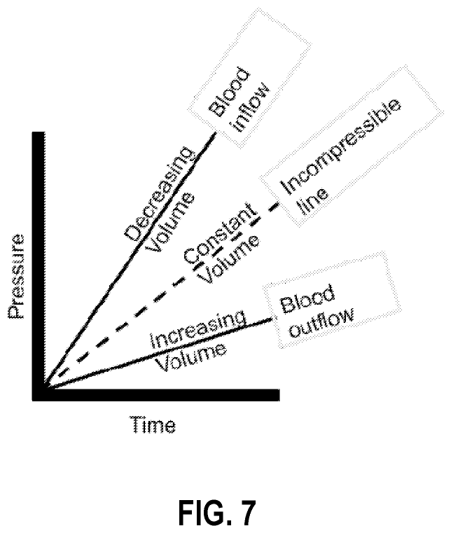

The slope of the incompressible line defines a ‘baseline’ pressure rise for a time unit. In the incompressible region the limb and bladder volumes are fixed. If the system volume is unchanged then the pressure should increase by this amount in each time unit. If blood flows out of the limb bladder volume increases. The increasing volume of the bladder will result in a lower pressure increase in a unit of time compared to the incompressible line. If blood flows into the limb, bladder volume decreases and pressure will increase faster than the incompressible line in a unit time as shown in FIG. 7. The incompressible line characterizes the system at an empirical level and thereby overcomes measurement issues related to patient limb size, bladder volume, and elasticity of limb and bladder. The pressure versus time relationship from the incompressible line allows measurements to be captured during the compression of the limb. This design allows the venous testing system to capture fast acting response to pressure stimulation. The characterization is relative which compensates for diversity in patients of limb size.

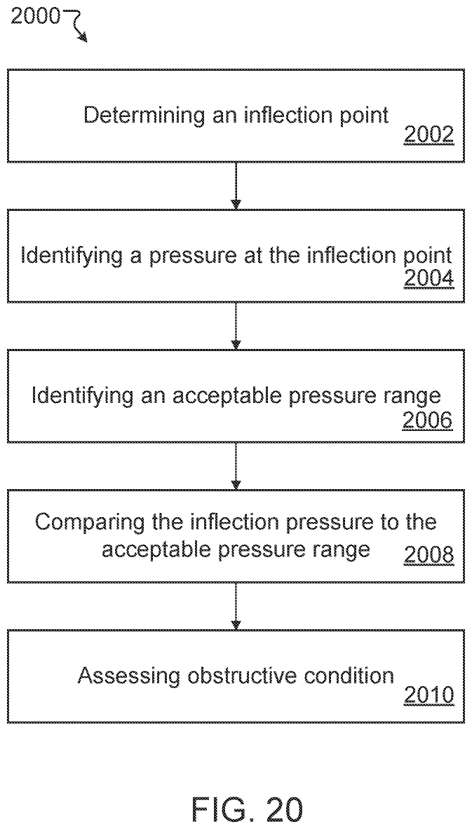

FIG. 20 is a flowchart of an example process 2000 for assessing obstruction in a venous or other anatomical system of a patient. The system obtains a pressure response curve from a compression cycle and identifies the upper inflection point (2002). The pressure at the inflection point is identified (2004). An acceptable pressure range can be defined, which may be standardized or determined based on patient characteristics such as sex, age, weight, height, BMI, and other factors. The acceptable range may have an upper and lower limit, for example. The system compares the inflection pressure to the acceptable pressure range (2008), and outputs an assessment about obstruction in the venous system (201) based on the comparison. If the inflection pressure is within the acceptable range, no obstruction may be identified. If the inflection pressure is above the upper limit and outside the acceptable range, obstruction may be identified. The system can output assessments of conditions such as obstruction in any number of ways, including presenting an indication of the assessment on a display unit, generating and delivering a report to a patient and/or clinician, storing the assessment in a memory of a computer system for later access, emailing a report of the assessment, or otherwise making a report availability to one or more users.

### Measuring the Amount of Blood Moved

Having a measure for the amount of blood moved out of the limb can be useful for several determinations. When the pressure measurements start to conform to the ‘incompressible’ line the blood has been forced out of the limb. Measuring from the beginning of compression to this point provides a measure of the amount of blood in the limb before the compression.

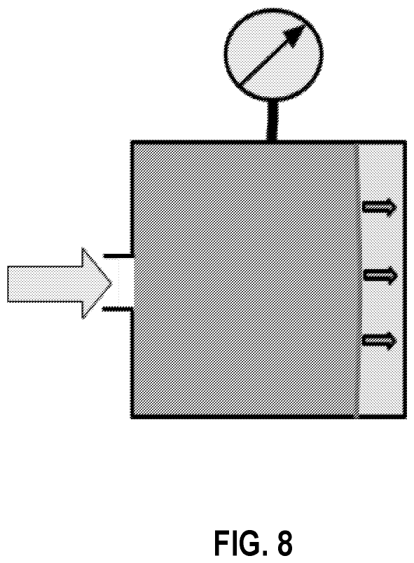

FIG. 8 illustrates how the xlength calculation is made. If air is injected at a constant rate into the container and the container expands at the same rate, pressure in the container will not increase. If air is injected and the container volume is constant then the pressure will rise at a linear rate. The rate is approximately linear over a small pressure change for a constant sized container. If air is added, the container expands and a pressure will increase is noted then some of the added air expanded the container and some caused a pressure increase. So the amount of air which caused the pressure increase can be subtracted and the remaining amount of air added would equal the container expansion.

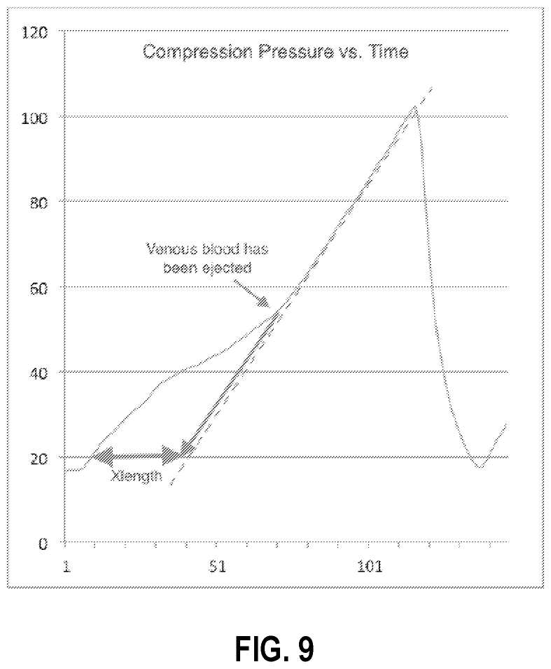

The compression stroke forces blood out of the limb until the upper inflection point. Most of the blood moves between 30 mmHg and the upper inflection point. This can be observed by the low slope in the active-flow/compressible region of the pressure response curve. With a constant volume rate for the pump in the apparatus and a known pressure to time relationship for the container (the bladder), the relative volume moved can be calculated. If no pressure rise occurred when the inflection point was reached the volume moved would be equal to the time. The pressure increase reached by the inflection point reflects the amount of added air devoted to raising pressure in the bladder. If this pressure raising air is subtracted the relative amount of blood moved is the time. The incompressible line represents the pressure to time relationship. Projecting the incompressible line down to the starting pressure can provide the ending time. The start time is the time at the measured starting pressure. The incompressible line provides the adjustment for the pressure rise by extending the line down to the pressure for the start of the measurement (see FIG. 9).

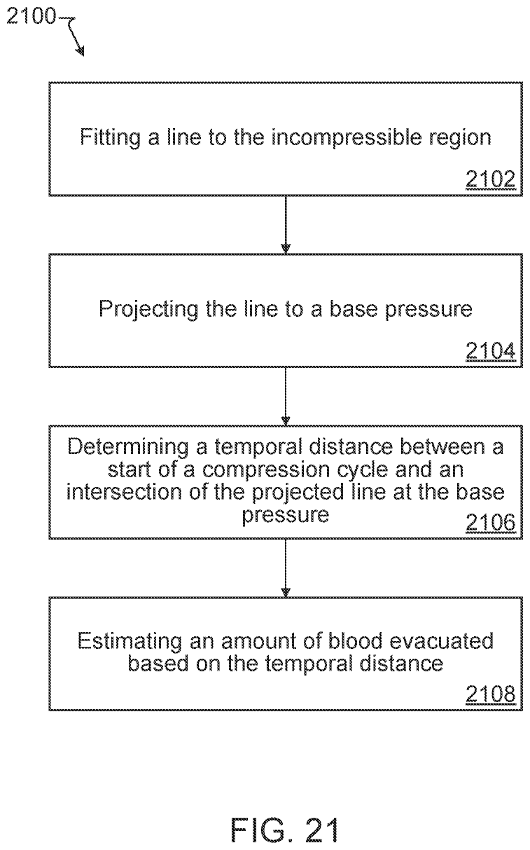

FIG. 21 is a flowchart of an example process 2100 for determining an amount of blood evacuated from a limb during a compression cycle according to the xlength calculation technique. The system obtains a pressure response curve from a compression cycle and identifies the incompressible region of the curve. Using any suitable technique (e.g., linear regression), the system fits a line to the incompressible region (2102) and projects the incompressible line to a base pressure (2104). The base pressure can be, for example, the pressure that existed at the start of the compression cycle (e.g., immediately before compression begins or immediately after beginning compression). The temporal distance (time interval) between the time associated with the base pressure (e.g., the time at the start of the compression cycle) and the time where the projection of the fitted incompressible line intersects the base pressure is determined (2108). The system then converts the temporal distance to a blood estimate based on the amount of air that was injected into the bladder over this time interval. For example, if the pump injects air into the bladder at a continuous rate, the rate of injection can be multiplied by the temporal distance to obtain a volume of air added to the bladder over this period of time. The same volume of blood can be assumed to be displaced by the volume of injected air over the temporal distance. In some implementations, a pre-defined relationship between air injection and blood displacement can be applied to estimate the amount of evacuated blood if, for example, the limb is modeled such that there is not 1:1 correspondence between the two.

### Measuring Obstruction

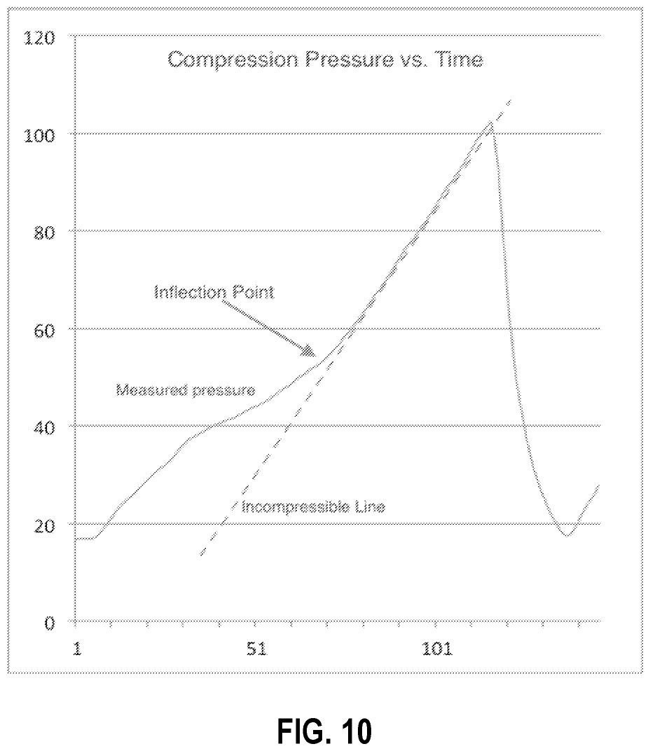

The pressure where the incompressible line begins, the inflection point, measures resistance to blood flow. In a seated patient the flow of venous blood out of the limb can be impeded by gravity. In the absence of venous obstruction the blood in the limb should be forced out at a pressure near the column height of the blood. Blood flows in the venous system with little resistance so the pressure required to force out the blood is equivalent to the height of blood from calf to shoulder. The inflection point occurs when the venous blood has been forced out of the limb. A venous obstruction prevents free flow of blood out of the limb. Forcing fluid through an obstruction causes an elevation in the pressure ahead of the obstruction. The elevation can be detected as an inflection point above the expected column height. FIG. 10 shows an inflection point for an unobstructed limb.

In one example, the inflection point is defined as the point where 85% of the venous blood that was in the calf prior to the start of the test has been moved out of the calf. It has been termed that the pressure at which the inflection point occurs is a patient's normal venous pressure. It has been established that a healthy patient's normal venous pressure at the inflection point is approximately 40 to 50 mmHg.

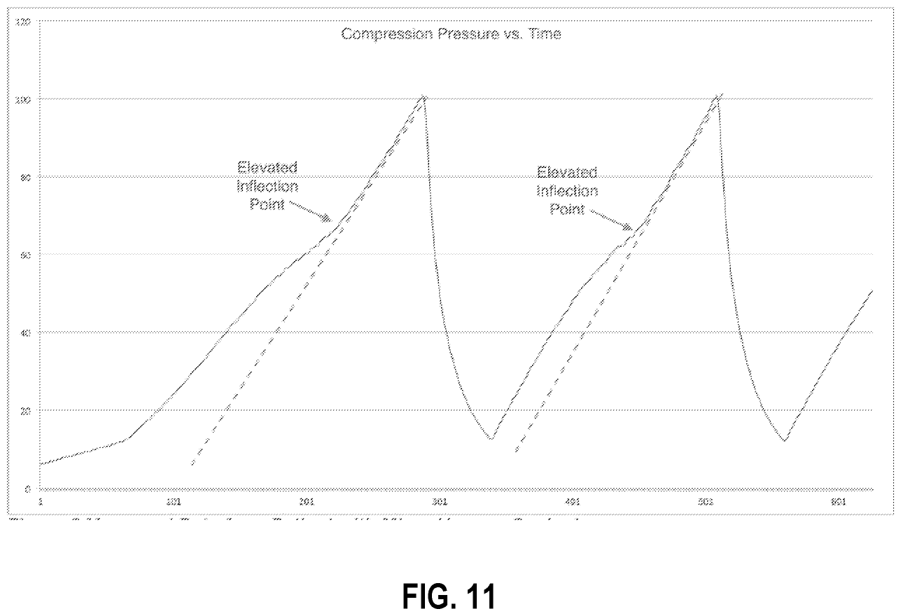

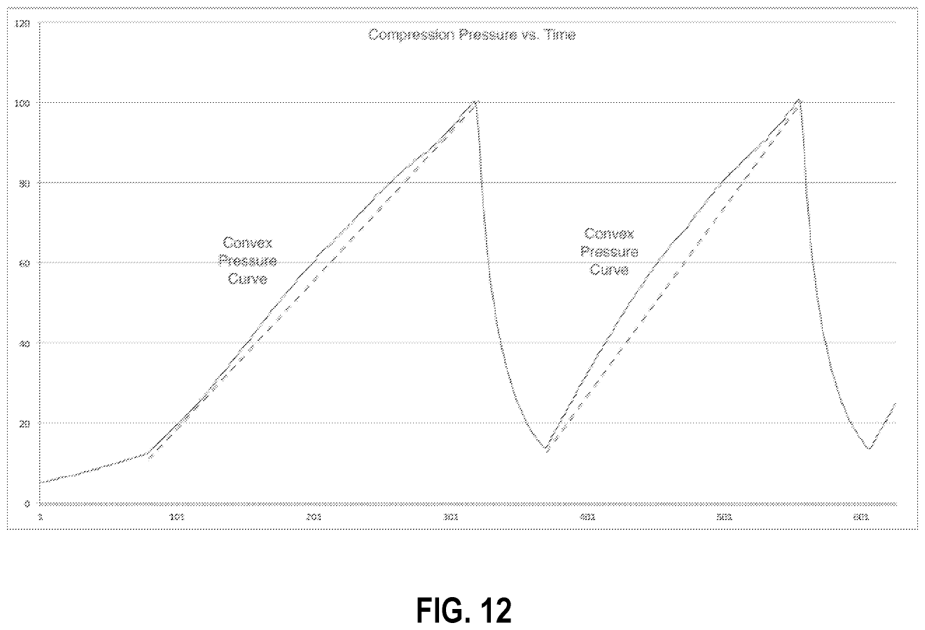

FIG. 11 shows measurements for a patient with venous occlusion. The inflection points (approximately 70 mmHg) are quite high. With significant obstruction the measured line becomes convex such that an incompressible line cannot be defined. Note that the rate of air volume injected into the bladder must be similar to the rate of healthy venous blood flow out of the limb. Too high a rate causes a false elevation in pressures by attempting to move blood at an excessive rate while too low a rate causes a very low elevation above normal if occlusion is present. FIG. 12 shows compression measurements for a patient with significant venous occlusion. The inflection points (approximately 90 mmHg) are very high and prevent the calculation of a reliable incompressible line.

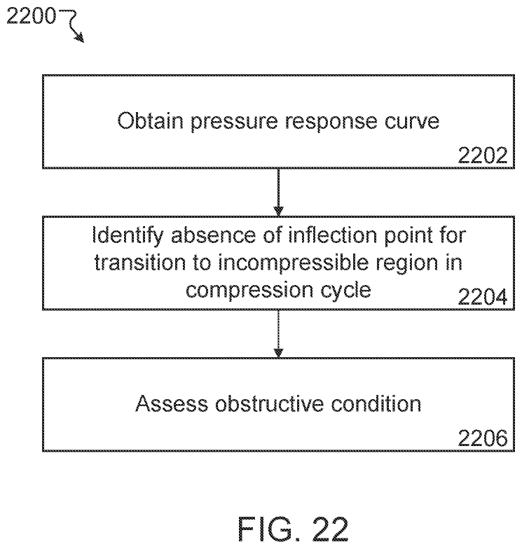

FIG. 22 is a flowchart of an example process 2200 for detecting obstruction in a venous system of a patient. The system obtains a pressure response curve from continuous pressure measurements during a compression cycle (2202). The system analyzes the pressure response curve to determine whether the curve exhibits a convex shape such that the curve lacks distinct pre-flow, reduced slope active flow, and increased slope post-flow regions (and thus lacks of an upper inflection point) (2204). If the pressure response curve determines that the curve exhibits the convex shape, the system generates and outputs an assessment that the venous system likely includes an obstruction blocking blood flow in the limb (2206).

### Compression Detection of Venous Insufficiency

The venous valves control the direction of blood flow. Flow should be out of the leg and up into the torso to return to the heart. If a valve fails, blood will flow backward into the lower leg causing the blood to pool leading to issues related to venous insufficiency. Valve failure can be observed during a compression which follows a decompression by detecting blood inflow as compression is applied. An initial compression occurs forcing the blood out of the limb. The compression is released and gravity will force blood into the limb through a failed valve. The inflow is detected during a subsequent compression by a pressure rise faster than the incompressible line, signaling an increase in the limb volume. The compression is not causing the increase in limb size but is detecting the increase due to the time at which it executes.

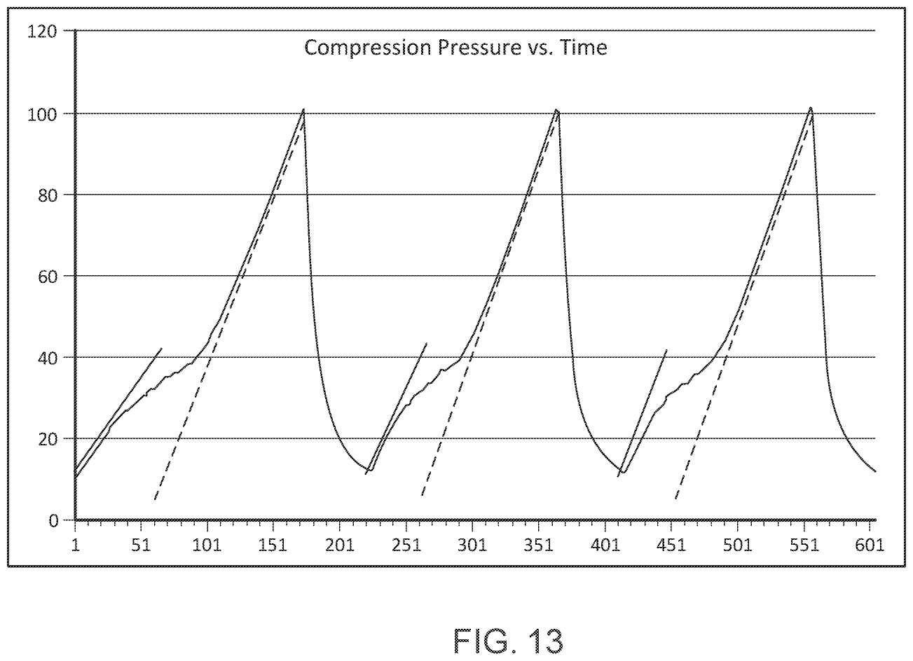

FIG. 13 shows the compression data for a patient with venous insufficiency. Note the convex nature of the initial section of the curve with regions where the slope is as high or higher than the incompressible slope. A slope higher than the incompressible slope indicates the inflow of blood into the leg during the compression. Blood is flowing backward into the leg during the lower pressures due to the weight of blood and decompression of the limb.

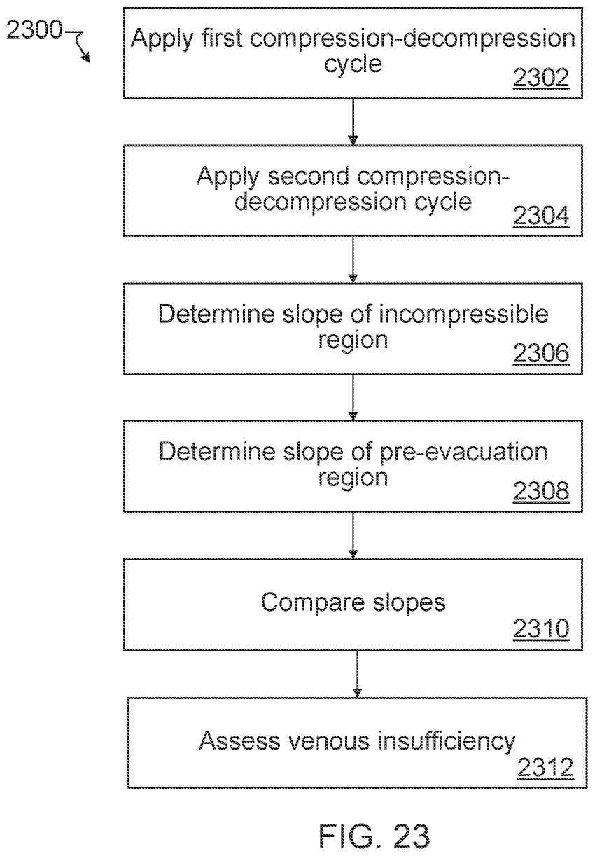

FIG. 23 is a flowchart of an example process 2300 for detecting venous insufficiency based on compression measurements. The controller of a venous testing system applies a first compression-decompression cycle to empty blood from the region of the patient limb under the cuff (2302). The controller then applies a second compression-decompression cycle (2304), and pressure measurements are continuously acquired during the cycle to obtain a pressure-response curve. The controller identifies the incompressible region of the curve for the second cycle and determines a slope of the incompressible region (2306). The system then determines a slope of the pre-flow (pre-evacuation) region of the compression phase of the second cycle and determines a slope of the pre-flow region (2308). The slopes of the incompressible region and pre-flow regions are compared (2310), and the system generates and outputs an assessment of venous insufficiency based on the comparison. If the slope is pre-flow region is greater than the slope of the incompressible region (or if the slope of the pre-flow region is greater than the slope of the incompressible region by at least a defined amount), then the system generates and outputs an assessment that patient likely suffers from venous insufficiency in the limb.

### Decompression Method to Detect Venous Insufficiency

Venous insufficiency involves valve failure allowing blood to flow backward in the system (away from the heart). In a seated patient gravity induces the backward flow. A compression stroke forces blood out of the limb and up into the torso. During the following decompression stroke blood would flow backward into the leg if not blocked by the valves. At pressure above column height blood inflow should be only arterial inflow. Below column height, blood flows into the limb from both retrograde flow and arterial inflow. Measuring reflow during a decompression stroke is the most direct way to detect retrograde flow. The decompression method relies on measurements taken during pauses in the decompression stroke.

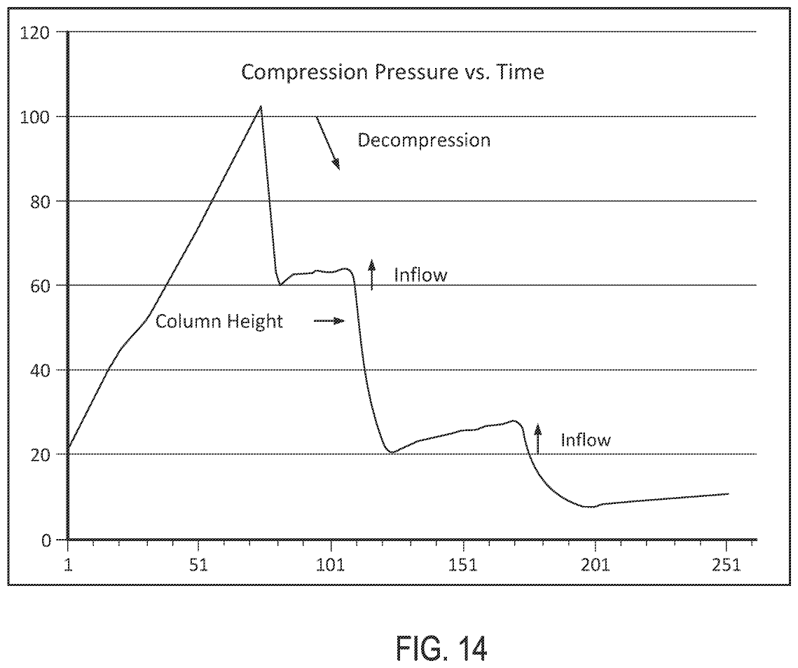

Measurements during a decompression stroke can be complicated by several factors. Venting air through a valve is nonlinear with much less air flowing at lower pressures due to the lower pressure differential between bladder and room pressure. Also, venting tends to be slow and blood will move quickly once compression pressure is below column height pressure. To correct for these issues the decompression is carried out in steps. Air is vented from the bladder immediately following the compression but only to a pressure above the column height (see FIG. 14). This step holds blood from flowing into the limb while reducing the air in the bladder so the next pressure drop can execute quickly after the pause interval in passive mode. The next step vents air from the cuff to a pressure significantly below column height to create a large impetus for blood to flow backward if valve failure exists in the venous system. Also, this range of pressure drop can be accomplished quickly so that the blood movement does not overlap with the pressure exhaust. After each pressure drop is executed, the valve is closed and pressure monitored. A rise in pressure indicates blood flow into the limb from arterial and/or retrograde blood flow.

The first pause above column height pressure shows a rise which is due to arterial inflow. This rise at higher pressure can be used for comparison to later increases to separate arterial inflow from retrograde flow. The main pressure drop occurs around 20 mmHg to provide a large differential and a pressure where the bladder couples well to the calf and measurements contain less noise. Below 20 mmHg pressure venting occurs quite slowly. The final decompression pause occurs at below 20 mmHg both to support a next compression and to capture any reflux that occurs at low pressures.

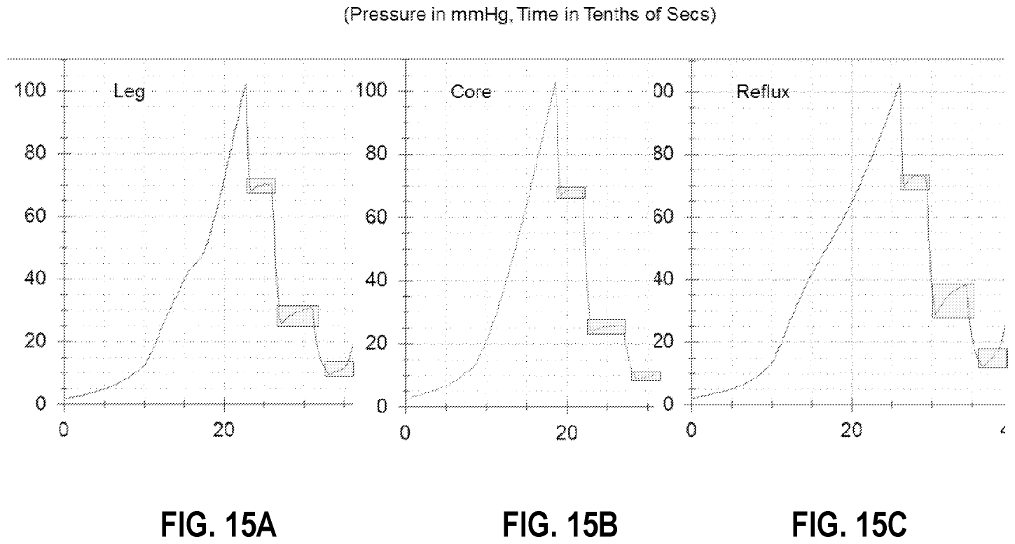

In FIG. 15, the plot labeled “Leg” shows a healthy leg. The plot labeled “Core” is with the pressure cuff wrapped around a rigid plastic tube with radius of a typical leg—note the lack of an inflection point due to the fact that there is no volume change in the plastic tube's radius when the cuff is compressed. The plot labeled “Reflux” shows a simulation of venous insufficiency.

The healthy “Leg” plot in FIG. 15 shows minimal pressure elevation after each decompression pause indicting a small inflow of blood into the leg's venous system. When compared to the “Core” plot, the paused regions between 20 and 40 mmHg and between 0 and 20 mmHg in the “Leg” plot do show some venous reflux. This is because the applied pressure at these points is below normal venous pressure (around 45 mmHg—the inflection point). Below normal venous some pressure backward flow can occur even in healthy legs. The “Leg” and “Core” plots in the paused region between 60 and 80 mmHg are very similar. In the “Leg” plot there is a very small change in the leg's volume, mostly due to the small amount of arterial flow into the leg. The “Reflux” plot in FIG. 13 shows a much larger inflow of blood into the leg's venous system during all three decompression pauses which indicates valve failure.

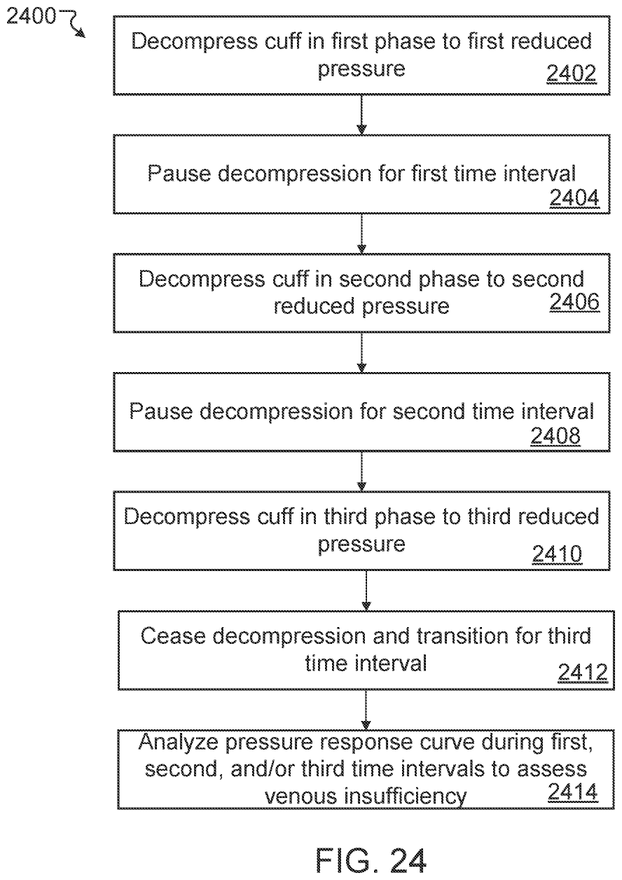

FIG. 24 is a flowchart of an example process 2400 for detecting venous insufficiency based on pressure measurements during a controlled, stepped decompression cycle. Following a compression cycle, the system partially decompresses the cuff in a first phase to first reduced pressure (2402). Pressure measurements are acquired continuously to obtain a pressure response curve over the entire process 2400. The first reduced pressure just above the column height. The system pauses decompression (e.g., transitions to passive mode) for a period of time (2404), then further decompresses the cuff in a second phase to a second reduced pressure significantly below column height (2406). At the second reduced pressure, decompression is again paused for a second time interval (2408). After the second time interval, decompression continues in a third phase to a third reduced pressure until the decompression cycle is completed (2410), and the system transitions into passive mode or another compression cycle (2412). The pressure response curve following each decompression phase can be analyzed to assess venous insufficiency (2414). For example, if the slopes of the pressure rises that occurred during the first, second, and/or third time interval are greater than a baseline amount (e.g., level, speed, or acceleration), the system can generate an output an assessment of venous insufficiency.

### Empty Leg Measurement

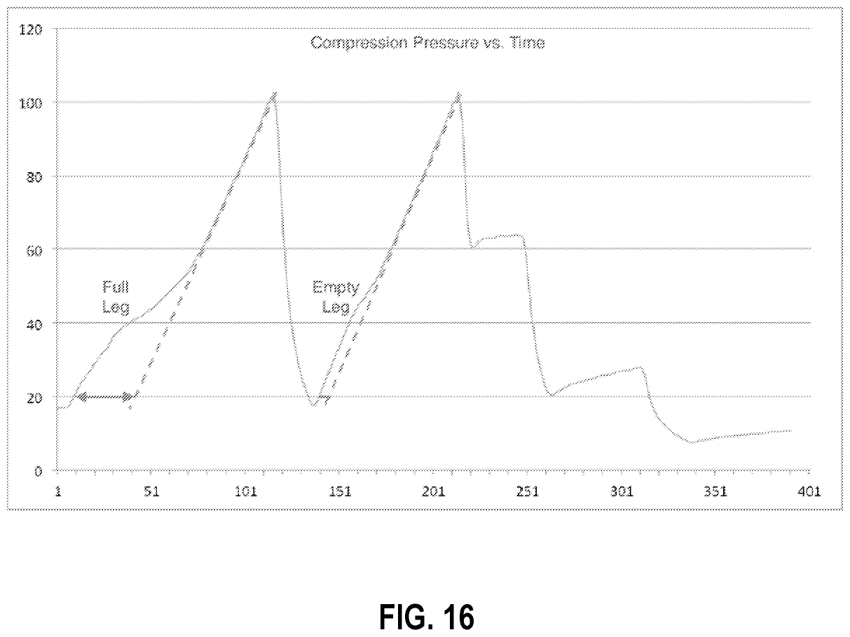

An empty leg compression stroke can be achieved by limiting the decompression in the cycle immediately beforehand. At the end of a compression stroke the limb should be substantially empty of blood. Refilling via arterial flow or venous reflux occurs during the following decompression stroke. If another compression is started before the leg can refill, the second compression executes on a mostly empty leg. The first compression stroke executes then decompression drops directly to (˜25-15 mmHg). Another compression stroke begins immediately. The goal is to execute back-to-back compressions with minimal time for refill in the leg. The first compression should have forced nearly all the blood from the calf. FIG. 16 illustrates an empty leg sequence of compressions. The blood amount moved in the second compression stroke should be equivalent to the blood moved had the leg been elevated above the torso. The measurements from the empty leg compression stroke are used in evaluation of pump function ejection.

### Measuring Pump Function

Movement of the calf muscles should pump blood out of the calf and toward the heart. Pump function is evaluated through patient exercise of the calf and xlength measurements. Pump function is measured by executing a sequence of compressions and comparing the amount of blood ejected under each condition. The amount of blood ejected during each compression is a relative measurement. A compression stroke is executed on a full leg during the cycle testing for obstruction. Because the patient is rested before this compression this amount is the maximum blood held in the leg. Other compressions can be executed to measure blood moved after pumping and for an ‘empty’ leg. The measured pump function outflow is evaluated on a continuum from a full leg amount to an empty leg amount. Good pump function nears an empty leg while poor function has an amount similar to the full leg. Pump function effectiveness is evaluated by the fraction of the blood moved on the scale from a full leg to an empty leg.

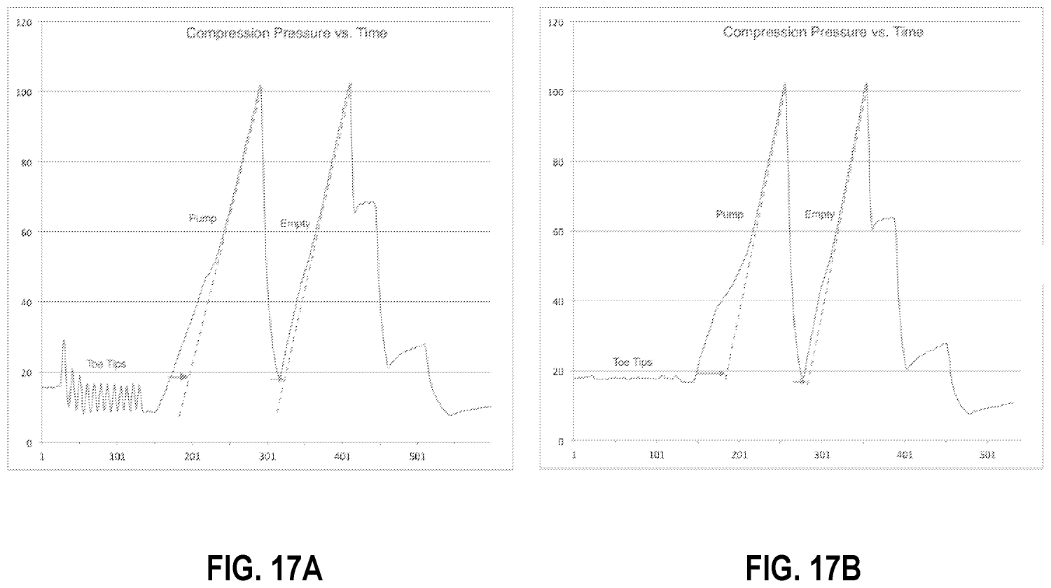

To measure the amount of blood moved after pumping, the patient is rested for a time to allow the leg to refill. Then the patient performs muscle movements consistent with pumping blood out of the leg (toe tips). Immediately after the toe tips finish a compression stroke is executed. The compression stroke should show a reduced blood outflow from the limb if muscle pumping is effective. FIGS. 17A and 17B depict pressure response curves for good and poor pump function, respectively.

### Compressions in Sequence

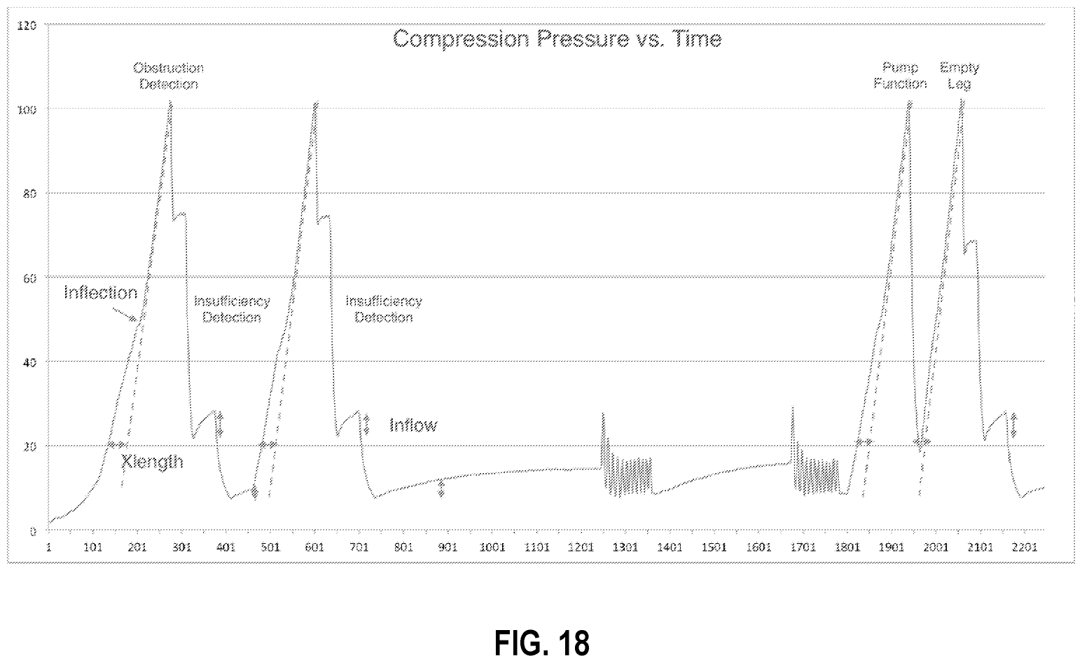

FIG. 18 depicts an extended plot of a pressure response curve over multiple compression-decompression and hold cycles of a testing protocol. The first compression stroke is intended to measure the maximum fullness of the calf (xlength) and inflection pressure. Inflection pressure detects and quantifies obstruction. Later compression strokes also provide an inflection pressure but the fullness of the first compression stroke provides a test performed with a large amount of blood. The decompression strokes provide measurement of inflow in order to detect and quantify venous insufficiency. The decompression stroke before the empty leg compression stroke is truncated to avoid inflow before the compression stroke. Inflow data from the other decompression strokes provides redundancy allowing measurement with noise to be ignored. Toe tips executed by the patient before the pump function compression stroke demonstrate how well the pump function empties the calf. The xlength measurement from the pump function compression stroke is evaluated on the scale of xlength from the full leg initial compression to the empty leg compression.

### Example Architecture of a Testing System

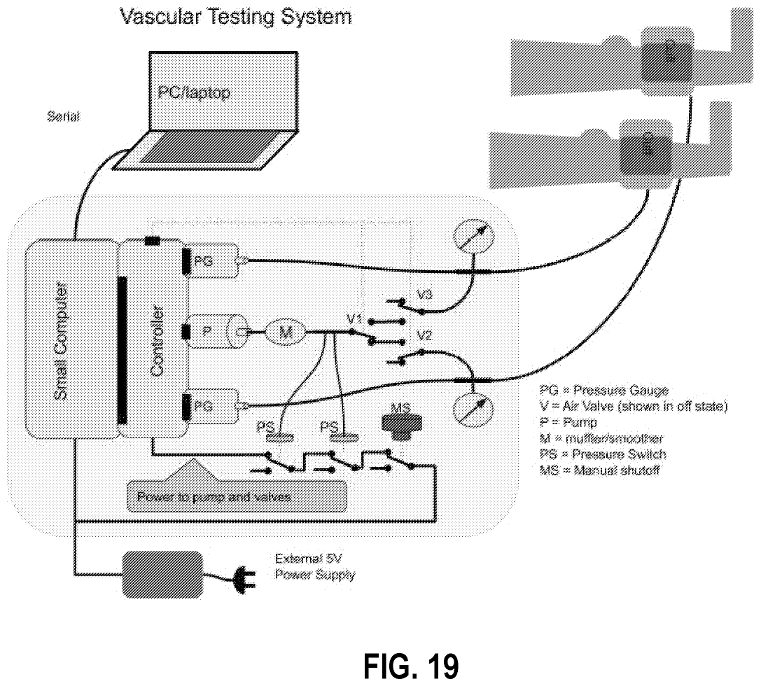

An example venous testing system is shown in FIG. 19, and includes tubing, pressure gauges, valves, an air pump, and a control/capture system. In this embodiment, the venous test system connects to an air bladder inside an inelastic cuff wrapped around the patient's limb. Air is added to or removed from the bladder at a controlled rate while monitoring the change in air pressure in the bladder. Valves control the flow of air into or out of the bladder in the cuff. The air pump supplies a constant volume of air to inflate the air bladder. Since the rate of air added to the bladder is constant, volume flow into the bladder equates to time. Pressure gauges measure pressure in the bladder. The control system captures pressure measurements while controlling the sequence of inflations and deflations. The system can control and measure one or multiple bladders on one or many limbs.

The venous test system does not require measurement of the absolute rate of volume introduced into the cuff, nor the volume of the cuff. An algorithm calibrates the measurements for the effects of cuff volume and limb size in the measured pressure curve by comparing to the incompressible regions.

The pathology of the limb creates pressure regions where the limb is nearly incompressible. The measurement of pressure versus time in the incompressible region provides a baseline for evaluating pressure versus time in other regions. Time is equivalent to added volume if the pump runs at a constant speed during a compression cycle. If the volume of the limb is unchanged, the volume of the bladder is unchanged and the pressure increase per time is constant. If the limb is contracting then bladder volume increases and less pressure per time is accumulated in the bladder. If the limb is expanding during a compression stroke the bladder will shrink and more pressure will be accumulated per time than the incompressible line describes.

The pressure measurements are captured during inflation so the venous test system requires the continuous, accurate measurement of pressure while pumping air into the cuff's bladder. Inflation and deflation operate concurrently with pressure measurement. Pulsatile noise produced by the pump during inflation can interfere with accurate pressure measurements. The venous test system can be constructed to overcome the issue of pulsatile noise by several methods. A ‘muffler’ can be introduced in the output path of the pump to smooth the flow from the pump before sending the pressure to the cuff's bladder. Alternatively, a bladder with two ports can be constructed where one port is used for inflation/deflation while the other port is connected to the pressure sensor. Similarly the pressure sensor can be positioned at the cuff's bladder. A baffle may be used to further reduce noise. Finally, a cuff design containing two bladders, one layered over the other, can be utilized where one bladder is initially inflated to a fixed volume and then used for sensing pressure (sense bladder) and a second bladder is layered over the first and inflated/deflated to apply/release compression pressure (drive bladder).

In some implementations, the system assesses venous occlusion by applying timed compressions and decompressions to a limb and observing the pressure with a model of how pressures-volume relationship will be affected by various venous issues. The system can also measure occlusion in the veins by applying timed compressions and decompressions to the limb and observing the pressure with a model of how pressures-volume relationship will be affected by various venous issues.

In some implementations, the system assesses venous incompetence by measuring reflux of blood by applying timed compressions and decompressions to the limb and observing the pressure with a model of how pressures-volume relationship will be affected by various venous issues.

In some implementations, the system measures pump function by comparing measurements in conjunction with muscle movement initiated by the patient by applying timed compressions and decompressions to the limb and observing the pressure with a model of how pressures-volume relationship will be affected by various operation.

### Computing and Processing Environment

Aspects of the subject matter and the actions and operations described in this specification can be implemented in digital electronic circuitry, in tangibly-embodied computer software or firmware, in computer hardware, including the structures disclosed in this specification and their structural equivalents, or in combinations of one or more of them. For example, the one or more controllers of the venous testing system can be implemented as or in one or more computer programs, e.g., one or more modules of computer program instructions, encoded on a computer program carrier, for execution by, or to control the operation of, data processing apparatus. The carrier can be a tangible non-transitory computer storage medium. Alternatively or in addition, the carrier can be an artificially-generated propagated signal, e.g., a machine-generated electrical, optical, or electromagnetic signal, that is generated to encode information for transmission to suitable receiver apparatus for execution by a data processing apparatus. The computer storage medium can be or be part of a machine-readable storage device, a machine-readable storage substrate, a random or serial access memory device, or a combination of one or more of them. A computer storage medium is not a propagated signal.

The term “data processing apparatus” encompasses all kinds of apparatus, devices, and machines for processing data, including by way of example a programmable processor, a computer, or multiple processors or computers. Data processing apparatus can include special-purpose logic circuitry, e.g., an FPGA (field programmable gate array), an ASIC (application-specific integrated circuit), or a GPU (graphics processing unit). The apparatus can also include, in addition to hardware, code that creates an execution environment for computer programs, e.g., code that constitutes processor firmware, a protocol stack, a database management system, an operating system, or a combination of one or more of them.

A computer program can be written in any form of programming language, including compiled or interpreted languages, or declarative or procedural languages; and it can be deployed in any form, including as a stand-alone program, e.g., as an app, or as a module, component, engine, subroutine, or other unit suitable for executing in a computing environment, which environment may include one or more computers interconnected by a data communication network in one or more locations.

A computer program may, but need not, correspond to a file in a file system. A computer program can be stored in a portion of a file that holds other programs or data, e.g., one or more scripts stored in a markup language document, in a single file dedicated to the program in question, or in multiple coordinated files, e.g., files that store one or more modules, sub-programs, or portions of code.

The processes and logic flows described in this specification can be performed by one or more computers executing one or more computer programs to perform operations by operating on input data and generating output. The processes and logic flows can also be performed by special-purpose logic circuitry, e.g., an FPGA, an ASIC, or a GPU, or by a combination of special-purpose logic circuitry and one or more programmed computers.

Computers suitable for the execution of a computer program can be based on general or special-purpose microprocessors or both, or any other kind of central processing unit. Generally, a central processing unit will receive instructions and data from a read-only memory or a random access memory or both. The essential elements of a computer are a central processing unit for executing instructions and one or more memory devices for storing instructions and data. The central processing unit and the memory can be supplemented by, or incorporated in, special-purpose logic circuitry.

Generally, a computer will also include, or be operatively coupled to, one or more mass storage devices, and be configured to receive data from or transfer data to the mass storage devices. The mass storage devices can be, for example, magnetic, magneto-optical, or optical disks, or solid state drives. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device, e.g., a universal serial bus (USB) flash drive, to name just a few.

To provide for interaction with a user, the subject matter described in this specification can be implemented on one or more computers having, or configured to communicate with, a display device, e.g., a LCD (liquid crystal display) monitor, or a virtual-reality (VR) or augmented-reality (AR) display, for displaying information to the user, and an input device by which the user can provide input to the computer, e.g., a keyboard and a pointing device, e.g., a mouse, a trackball or touchpad. Other kinds of devices can be used to provide for interaction with a user as well; for example, feedback and responses provided to the user can be any form of sensory feedback, e.g., visual, auditory, speech or tactile; and input from the user can be received in any form, including acoustic, speech, or tactile input, including touch motion or gestures, or kinetic motion or gestures or orientation motion or gestures. In addition, a computer can interact with a user by sending documents to and receiving documents from a device that is used by the user; for example, by sending web pages to a web browser on a user's device in response to requests received from the web browser, or by interacting with an app running on a user device, e.g., a smartphone or electronic tablet. Also, a computer can interact with a user by sending text messages or other forms of message to a personal device, e.g., a smartphone that is running a messaging application, and receiving responsive messages from the user in return.

In this specification, the term “database” refers broadly to refer to any collection of data: the data does not need to be structured in any particular way, or structured at all, and it can be stored on storage devices in one or more locations. Thus, for example, the index database can include multiple collections of data, each of which may be organized and accessed differently.

Similarly, in this specification the term “engine” refers broadly to refer to a software-based system, subsystem, or process that is programmed to perform one or more specific functions. Generally, an engine will be implemented as one or more software modules or components, installed on one or more computers in one or more locations. In some cases, one or more computers will be dedicated to a particular engine; in other cases, multiple engines can be installed and running on the same computer or computers.

This specification uses the term “configured to” in connection with systems, apparatus, and computer program components. That a system of one or more computers is configured to perform particular operations or actions means that the system has on it software, firmware, hardware, or a combination of them that in operation cause the system to perform the operations or actions. That one or more computer programs is configured to perform particular operations or actions means that the one or more programs include instructions that, when executed by data processing apparatus, cause the apparatus to perform the operations or actions. That special-purpose logic circuitry is configured to perform particular operations or actions means that the circuitry has electronic logic that performs the operations or actions.

The subject matter described in this specification can be implemented in a computing system that includes a back-end component, e.g., as a data server, or that includes a middleware component, e.g., an application server, or that includes a front-end component, e.g., a client computer having a graphical user interface, a web browser, or an app through which a user can interact with an implementation of the subject matter described in this specification, or any combination of one or more such back-end, middleware, or front-end components. The components of the system can be interconnected by any form or medium of digital data communication, e.g., a communication network. Examples of communication networks include a local area network (LAN) and a wide area network (WAN), e.g., the Internet.

The computing system can include clients and servers. A client and server are generally remote from each other and typically interact through a communication network. The relationship of client and server arises by virtue of computer programs running on the respective computers and having a client-server relationship to each other. In some implementations, a server transmits data, e.g., an HTML page, to a user device, e.g., for purposes of displaying data to and receiving user input from a user interacting with the device, which acts as a client. Data generated at the user device, e.g., a result of the user interaction, can be received at the server from the device.

While this specification contains many specific implementation details, these should not be construed as limitations on the scope of what is being claimed, which is defined by the claims themselves, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially be claimed as such, one or more features from a claimed combination can in some cases be excised from the combination, and the claim may be directed to a subcombination or variation of a subcombination.

Similarly, while operations are depicted in the drawings and recited in the claims in a particular order, this by itself should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described above should not be understood as requiring such separation in all embodiments, and it should be understood that the described program components and systems can generally be integrated together in a single software product or packaged into multiple software products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In some cases, multitasking and parallel processing may be advantageous.

What is claimed is:

1. A method for assessing a fluid system of a patient, comprising:
applying, with a fluid evacuation system, compression to a cuff on a limb of the patient;
measuring a pressure of the cuff during the compression to obtain a pressure response curve;
analyzing the pressure response curve to identify an incompressible region, wherein the incompressible region is a portion of the pressure response curve indicative of a bodily fluid having been substantially evacuated from at least a portion of the limb as a result of the compression; and
using information about the incompressible region of the pressure response curve (i) to screen for one or more problems with the fluid system or (ii) to evaluate an efficacy of the fluid evacuation system in evacuating the bodily fluid from the limb of the patient.

2. The method of claim 1, wherein the limb is a leg of the patient, the cuff is disposed on a calf of the leg when the compression is applied, and the at least the portion of the limb comprises the calf.

3. The method of claim 1, wherein analyzing the pressure response curve to identify the incompressible region comprises identifying an inflection point in the pressure response curve that marks a transition from a compressible region of the pressure response curve to the incompressible region.

4. The method of claim 3, wherein the compression is applied to the cuff in a compression cycle, wherein the inflection point corresponds to a point in the compression cycle in which a predetermined amount of bodily fluid has been evacuated from the at least the portion of the limb.

5. The method of claim 3, comprising screening for an obstruction in the fluid system based on a pressure value at the inflection point.

6. The method of claim 3, comprising:
fitting a line to the incompressible region of the pressure response curve; and
determining, using the line, an amount of bodily fluid evacuated from the at least the portion of the limb during the compression.

7. The method of claim 1, comprising screening for an obstruction in the fluid system based on an absence in the pressure response curve of an inflection point between a compressible region of the pressure response curve and an incompressible region of the pressure response curve.

8. The method of claim 1, wherein the compression is applied to the cuff in a first compression-decompression cycle, and the method comprises:
applying compression to the cuff in a second compression-decompression cycle following the first compression-decompression cycle;
determining a slope of a second incompressible region in the pressure response curve, wherein the second incompressible region occurs during compression in the second compression-decompression cycle;
determining a slope of a pre-evacuation region in the pressure response curve, wherein the pre-evacuation region precedes the second incompressible region during compression in the second compression-decompression cycle;
comparing the slope of the pre-evacuation region to the slope of the second incompressible region; and
assessing vessel insufficiency based on a result of comparing the slope of the pre-evacuation region to the slope of the second incompressible region.

9. The method of claim 1, comprising:
applying decompression to the cuff following the compression, including:
decompressing the cuff in a first phase from a peak pressure to a first reduced pressure that is less than a defined amount of column height pressure;
pausing, for a first paused interval, the decompression after the first phase upon reaching the first reduced pressure;
decompressing the cuff in a second phase from the first reduced pressure to a second reduced pressure that is below the column height pressure;
pausing, for second paused interval, the decompression after the second phase upon reaching the second reduced pressure;
decompressing the cuff in a third phase from the second reduced pressure to a third reduced pressure;
measuring the pressure of the cuff during the decompression, including during the first phase, the second phase, and the third phase, and augmenting the pressure response curve with a pressure response measured during the decompression; and
assessing vessel insufficiency based on characteristics of the pressure response curve during at least one of the first paused interval or the second paused interval.

10. The method of claim 1, wherein the compression is applied to the cuff in a first compression-decompression cycle, and the method comprises:
applying compression to the cuff in a second compression-decompression cycle following the first compression-decompression cycle, wherein the compression in the second compression-decompression cycle is initiated before the bodily fluid substantially refills in the limb;
measuring the pressure of the cuff during the compression in the second compression-decompression cycle, and augmenting the pressure response curve with a pressure response measured during the compression in the second compression-decompression cycle; and
assessing ejection function of a heart of the patient based on at least one characteristic of the pressure response measured during the compression in the second compression-decompression cycle.

11. The method of claim 1, wherein applying compression to the cuff comprises pumping a working fluid into the cuff at a constant rate, wherein the working fluid is compressed air, oxygen, or water.

12. The method of claim 1, wherein the fluid system is a venous system and the fluid is blood, the fluid system is an arterial system and the fluid is blood, or the fluid system is a lymphatic system and the fluid is lymph.

13. One or more computer-readable storage media encoded with instructions that, when executed by one or more computers, cause the one or more computers to perform opera rations comprising:
applying with a fluid evacuation system, compression to a cuff on a limb of the patient,
measuring a pressure of the cuff during the compression to obtain a pressure response curve;
analyzing the pressure response curve to identify an incompressible region, wherein the incompressible region is a portion of the pressure response curve indicative of a bodily fluid having been substantially evacuated from at least a portion of the limb as a result of the compression; and
using information about the incompressible region of the pressure response curve (i) to screen for one or more problems with the fluid system or (ii) to evaluate an efficacy of the fluid evacuation system in evacuating the bodily fluid from the limb of the patient.

14. (canceled)

15. A system, comprising:
a pump configured to increase pressure of a working fluid in a cuff during a compression action;
a valve configured to release pressure from the cuff during a decompression action;
a pressure transducer configured to measure a pressure of the working fluid in the cuff; and
one or more controllers configured to actuate the pump and the valve to control a level of pressure of the working fluid in the cuff via compression and decompression actions based at least in part on measurements from the pressure transducer.

16. The system of claim 15, further comprising the cuff.

17. The system of claim 15, further comprising a flow meter configured to measure a flow of the working fluid into or out of the cuff, wherein the one or more controllers are further configured to control the level of pressure of the working fluid in the cuff based on measurements of working fluid flow from the flow meter.

18. The system of claim 17, further comprising one or more bodily fluid sensors configured for placement on a limb of a patient in a region of the limb separate from the cuff, wherein the system is further configured to measure a level of flow of a bodily fluid in the patient using signals from the one or more bodily fluid sensors, wherein the bodily fluid is blood or lymph.

19. (canceled)
